(19) United States
(12) Patent Application Publication
(10) Pub. No.: US 2016/0214926 A1
(43) Pub. Date: Jul. 28, 2016

(54) DIPHENYLOXYALKYLAMINE DERIVATIVES AND ARYLOXYALKYLAMINE DERIVATIVES, PHARMACEUTICAL COMPOSITION, USE OF SAID PHARMACEUTICAL COMPOSITION FOR TREATING, PREVENTING OR INHIBITING CHRONIC PULMONARY INFLAMMATORY DISEASES AND METHOD FOR TREATING OR PREVENTING SUCH DISEASES

(71) Applicant: FUNDACAO OSWALDO CRUZ, Rio de Janeiro, RJ (BR)

(72) Inventors: Marco Aurelio MARTINS, Rio de Janeiro (BR); Jorge Carlos Santos DA COSTA, Rio de Janeiro (BR); Emerson Teixeira DA SILVA, Rio de Janeiro (BR); Robson Xavier FARIA, Rio de Janeiro (BR); Marcus Vinicius Nora DE SOUZA, Rio de Janeiro (BR); Magda Fraguas SERRA, Rio de Janeiro (BR)

(21) Appl. No.: 14/911,138

(22) PCT Filed: Aug. 6, 2014
(86) PCT No.: PCT/BR2014/000266

§ 371 Date: Feb. 9, 2016

(30) Foreign Application Priority Data
Aug. 9, 2013 (BR) 102013 020313 0

(51) Int. Cl. C07C 217/20; C07C 233/25; C07C 217/80; C07C 217/14; C07C 217/18

(52) U.S. Cl. CPC C07C 217/20; C07C 217/14; C07C 217/18; C07C 217/80; C07C 233/25

(57) ABSTRACT

The present invention relates to diphenyloxyalkylamine derivatives and aryloxyalkylamine derivatives that are structurally analogous to mexiletine, said derivatives having important biological activity and not causing the undesired side effects observed with the prototype, as well as with other drugs from the same therapeutic class as the prototype. The derivatives of the present invention have formulas II and III and are used for treating, preventing or inhibiting pulmonary inflammatory diseases, for example, asthma and chronic obstructive pulmonary disease (COPD).

### APPLICATION FIELD

The present invention relates to diphenyloxyalkylamine derivatives and aryloxyalkylamine derivatives that are structurally analogous to mexiletine, with important biological activities. Such analogues showed higher relaxation potency of the respiratory smooth muscle and marked anti-inflammatory action in the lung tissue, compared to mexiletine prototype. Importantly, the derivatives of the present invention are devoid of undesirable side effects present in the prototype, as well as in other drugs of the same therapeutic class as the prototype.

The abovementioned derivatives, that are structurally analogous to mexiletine, are part of the pharmaceutical composition of the present invention, used in the form of free base or pharmaceutically acceptable salts thereof, preferably hydrochlorides.

The present invention contemplates also the use of the pharmaceutical composition as a medicine for the treatment and/or prevention of inflammatory lung diseases, such as asthma and chronic obstructive pulmonary disease (COPD).

Finally, the present invention provides a method of treating, preventing or inhibiting inflammatory lung diseases, such as asthma and COPD, comprising administering a pharmacologically effective amount of said pharmaceutical composition by any route of administration.

### BACKGROUND OF THE INVENTION

The mexiletine, 2-(2-aminopropoxy)-1,3-dimethylbenzene, represented in formula (I) below, registered with the trade name Mexitil®, is a counterpart of the local anesthetic lidocaine clinically used by mouth for controlling cardiac arrhythmias (Campbell, 1987) and relieving pain of different origins, including neuropathic pain (Jarvis et al., 1998) and cephalalgias difficult to treat (Marmura et al., 2008).

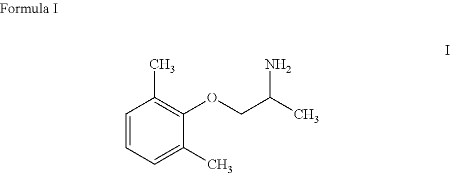

U.S. Pat. No. 3,659,019 and U.S. Pat. No. 3,954,872 describe the structure and use of mexiletine. Pharmaceutical formulations using mexiletine for controlling arrhythmias can be found in U.S. Pat. No. 4,031,244.

The mexiletine acts by inhibiting the propagation of action potential in Purkinje network with low interference on the autonomic nervous system, by blocking the fast sodium channels (Monk et al., 1990). Also orally, mexiletine is capable of inducing relaxation of airways in asthmatic patients, suggesting a potential, as the therapeutic use, in the pharmacological control of asthma (Groeben et al., 1996). However, this application comes up against important limitations arising from the very action of mexiletine as inhibitor of sodium channel, which is inherently linked to serious side effects such as cardiovascular toxicity as well as gastrointestinal and central disorders (Campbell, 1987).

The lungs play a central role in gas exchange and make direct connection with the external environment Because of this, allergic, infectious and occupational disorders of the respiratory system are among the most frequent and disabling diseases that affect humans (Saraiva et al., 2011). Asthma is characterized by non-specific bronchial hyperreactivity and marked eosinophilic inflammatory infiltrate in the lungs. Recurrent episodes of breathlessness, wheezing and cough are the main symptoms of this disease, which if not treated can cause death (Lemanske et al., 2010; Mannam et al., 2010). Recent data indicate that the number of asthmatics is increasing in the world. The disease affects people of all ages and kills six people every day in Brazil (Brightling et al., 2012). Chronic obstructive pulmonary disease (COPD) includes those patients who in general are affected simultaneously with emphysema and chronic bronchitis. Emphysema destroys the walls of the alveolar sacs, decreasing dramatically the surface area available for gas exchange. Bronchitis causes constriction of the pulmonary airways and blocks them with an exaggerated production of mucus (Brody, 2012). COPD is one of the biggest killers worldwide and is difficult to diagnose both in developed and developing nations (Dance, 2012).

Thus, it is highly desirable to develop substances which act in the treatment, prevention or inhibition of pulmonary inflammatory disorders, without the disadvantages indicated by the state of the art.

### SUMMARY OF THE INVENTION

The present invention, in its most general aspect, refers to derivatives structurally analogous to mexiletine, that is, diphenyloxyalkylamines and aryloxyalkylamines, with anti-inflammatory and bronchodilator properties. For its lower activity on sodium channels, there are indications that new diphenyloxyalkylamine derivatives and aryloxyalkylamine derivatives are devoid of the side effects present in other drugs in the same therapeutic class. More specifically, the invention relates to structural modifications in the mexiletine molecule, producing new derivatives containing structural unprecedented pattern and low activity on the sodium channel. Interestingly, these analogues are capable of inhibiting the contraction of respiratory smooth muscle, in addition to blocking the pulmonary inflammatory response triggered by various stimuli, including allergens and cigarette smoke.

An objective of the present invention is to provide a pharmaceutical composition containing at least one of the derivatives derived from the classes of diphenyloxyalkylamines and aryloxyalkylamines as active ingredient, or a combination of both.

Another objective of the present invention refers to the use of a pharmaceutical composition as a medicine for the treatment and/or prevention of inflammatory lung diseases, such as asthma and chronic obstructive pulmonary disease (COPD).

Another objective of the present invention relates to a method of treatment, prevention or inhibition of atopic diseases including asthma, COPD, rhinitis, allergic hives, chronic lung inflammation associated with eosinophilia, such as non-atopic asthma and chronic intestinal inflammation, such as colitis, comprising administering a pharmacologically effective amount of at least one of these compounds. Particularly, the present invention provides a method of treating, preventing or inhibiting inflammatory lung diseases, such as asthma and COPD, comprising administering a pharmacologically effective amount of said pharmaceutical composition by any route of administration.

In the present invention, all derivatives of the classes of diphenyloxyalkylamines and aryloxyalkylamines are presented in the form of free base or pharmaceutically acceptable salts thereof, preferably hydrochlorides.

### DESCRIPTION OF TABLES AND FIGURES

Table 1: Comparative effect of inhibition of sodium current evidenced by mexiletine and analogues, from the classes of diphenyloxyalkylamines and aryloxyalkylamines, in GH3 cells evaluated in the patch clamp system.

Table 2: Potency values (IC₅₀) and maximum effect (EMAX) of inhibition of the contraction response induced by carbachol (10 μM) on rat tracheal rings pretreated with mexiletine, JME-173 or JME-207, representatives of the class of aryloxyalkylamines. Data represent the mean±SEM from 4 to 7 tracheal rings.

Table 3: Comparative values of inhibition potency (IC₅₀) and maximum effect (EMAX) of mexiletine, JME-207, JME-173 and JME-209, of the classes of diphenyloxyalkylamines and aryloxyalkylamines, relative to the blocking of anaphylactic mast cell degranulation.

FIG. 1: Relaxing effect of the compounds aryloxyalkylamines JME-173 and JME-207, compared to that of mexiletine in trachea pre-contracted with carbachol.

FIG. 2: Antispasmodic effect of aryloxyalkylamine JME-173 (Part A) and salmeterol (Part B) in the anaphylactic contraction of tracheal rings.

FIG. 3: Mast cell stabilizing effect exhibited by compounds of the classes of diphenyloxyalkylamines and aryloxyalkylamines, JME-173, JME-207, JME-209, JME-141 and mexiletine.

FIG. 4: Antispasmodic effect of diphenyloxyalkylamine JME-209 (30 and 100 mg/kg, orally) or carrier (0.9% NaCl), evaluated in mice subjected to methacholine challenge.

FIG. 5: Antispasmodic effect of aryloxyalkylamine JME-207 (30 and 100 mg/kg, orally) or carrier (0.9% NaCl), evaluated in mice subjected to methacholine challenge.

FIG. 6: Protocol for sensitization, antigen challenge and treatment used to assess the activity of the tested compounds in the experimental asthma model in mice.

FIG. 7: Effect of nebulization of aryloxyalkylamines JME-141, JME-173, JME-188 or JME-207 on airway hyperresponsiveness in mice sensitized and challenged with ovalbumin.

FIG. 8: Effect of nebulization of aryloxyalkylamines JME-141, JME-173, JME-188 or JME-207 on leukocyte infiltration evaluated in bronchoalveolar lavage in mice sensitized and challenged with ovalbumin.

FIG. 9: Effect of nebulization of aryloxyalkylamines JME-141, JME-173, JME-188 or JME-207 on the generation of cytokines in lung tissue of mice sensitized and challenged with ovalbumin.

FIG. 10: Effect of nebulization of aryloxyalkylamine JME-173 (0.5-2%) on airway hyperresponsiveness in mice sensitized and challenged with ovalbumin.

FIG. 11: Effect of nebulization of aryloxyalkylamine JME-173 on the production of mucus in the airways in mice sensitized and challenged with ovalbumin.

FIG. 12: Effect of nebulization of aryloxyalkylamine JME-173 on the sub-epithelial fibrosis airway response in mice sensitized and challenged with ovalbumin.

FIG. 13: Effect of oral treatment with diphenyloxyalkylamine JME-209 on the increase of total count of leukocytes, mononuclear cells, eosinophils and neutrophils observed in bronchoalveolar lavage after stimulation with LPS in mice.

FIG. 14: Effect of oral treatment with diphenyloxyalkylamine JME-209 on lung hyperresponsiveness observed after stimulation with LPS in mice.

FIG. 15: Effect of oral treatment with diphenyloxyalkylamine JME-209 or dexamethasone on the increase of total count of leukocyte in bronchoalveolar lavage of mice exposed to cigarette smoke.

FIG. 16: Effect of oral treatment with diphenyloxyalkylamine JME-209 or dexamethasone on the increase of total count of mononuclear cells, neutrophils and eosinophils in bronchoalveolar lavage of mice exposed to cigarette smoke.

### DETAILED DESCRIPTION OF THE INVENTION

It has been observed by the inventors that suitable structural modifications in the mexiletine molecule result in obtaining analogues with anti-inflammatory and bronchodilator properties. An important aspect is that such derivatives have low activity on the sodium channel, unlike the prototype, as seen in electrophysiological assays using the “patch clamp” technique in GH3 cells. Based on these data, the present invention proposes a new therapeutic method for the treatment of diseases related to obstruction and inflammation of airways, such as asthma and COPD, by topical or systemic administration of diphenyloxyalkylamine derivatives and aryloxyalkylamine derivatives, devoid of local anesthetic and antiarrhythmic activity, such as diphenyloxyalkylamine derivatives and aryloxyalkylamine derivatives disclosed by this invention.

The diphenyloxyalkylamine derivatives and aryloxyalkylamine derivatives of the invention are characterized in that the compounds, or one of its salts formed by organic or mineral acids, are represented by the formulas (II) and (III) below:

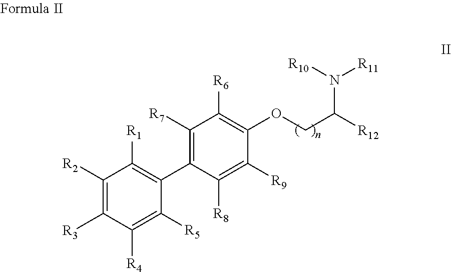

wherein
the substituents R1, R2, R3, R4, R5, R6, R7, R8, R9 and R12 may be characterized by one (or more) H, CH3, OH, CF₃, alkoxides, halogens, linear or branched and/or cyclic alkyl radicals, benzyl groups, phenyl, alkenes or alkynes, hydroxyl, hydroxyalkyl, thioalkyl or oxygen functions in acyclic or cyclic systems, forming the heterocyclic ring. Include electron donor and remover groups, as acetamide and the nitro group;
the substituents R10 and R11 may be represented by H, CH3, linear or branched and/or cyclic alkyl radicals, benzyl groups, phenyl, alkenes or alkynes, or oxygen functions in acyclic or cyclic systems, forming the heterocyclic ring.
“n” can be formed of 1 to 4 carbon atoms as spacer;

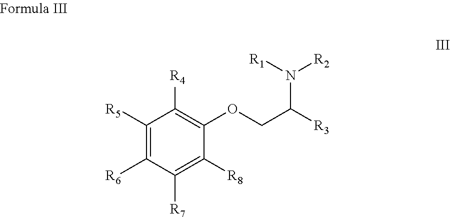

### Wherein:

the substituents R1 and R2 may be characterized by one (or more) H, CH3, linear or branched and/or cyclic alkyl radicals, benzyl groups, phenyl, alkenes or alkynes, oxygen functions in acyclic or cyclic systems, forming the heterocyclic ring;
the substituent R3 may be characterized by CH3, linear or branched and/or cyclic alkyl radicals, benzyl groups, phenyl, alkenes or alkynes;
R4, R5, R6, R7 and R8 may be represented by H, CH3, OH, linear or branched and/or cyclic alkyl radicals, benzyl groups, phenyl, alkenes or alkynes, ethers, thioethers, halogens, amines, and alkylamines. Include electron donor and remover groups. Electron donor and remover groups are defined in the description of preferred embodiments.
“n” can be formed of 1 to 4 carbon atoms as a spacer;

### DESCRIPTION OF THE PREFERRED EMBODIMENTS

The examples shown herein are intended only to exemplify, but without limiting the scope of the invention.

As used herein, the term alkyl means an alkyl group of linear, branched or cyclic chain, of up to eight (8) carbon atoms. Examples of alkyl groups used in the present invention are methyl, ethyl, propyl, butyl, “Alkyl ether”, i.e. alkoxy can be interpreted herein as alkyl group, e.g., methoxy, ethoxy.

As used herein, the term alkene means an alkene group of linear, branched or cyclic chain, of up to eight (8) carbon atoms. Examples of alkene groups used in the present invention are methylene, ethylene, propylene.

As used herein, the term cyclic alkyl means a cycle alkane, alkene or containing heteroatoms, e.g., oxygen or sulfur.

As used herein, “room temperature” includes a range of 20 to 35° C.;

As used herein, the term electron remover grouping includes nitro, cyano, azide, carbonyl, carboxyl, amidine, halogen groups;

As used herein, the term electron donor grouping includes methoxyl, ethoxyl, hydroxyl, alkylamine, amine groups.

Salts of compounds of formula II or III include acid salts, such as HCl and HBr. Preferred salts are those pharmaceutically acceptable. Salts of compounds of formula II or III correspond to pharmaceutically acceptable salts, including acid salts, such as HCl and HBr.

The preferred compounds of the present invention are defined by the following structures—Table I, among others:

TABLE I

| Structure | Code |
|---|---|
| 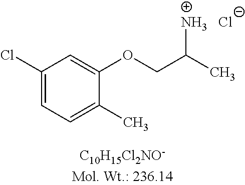 | JME-170 |
| 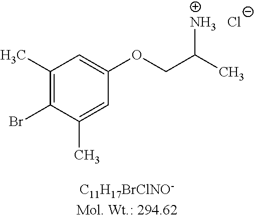 | JME-173 |
| 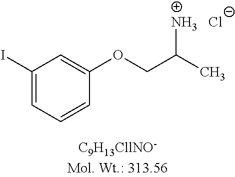 | JME-141 |
| 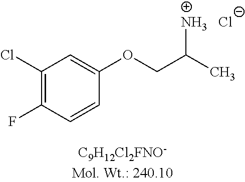 | JME-188 |
| 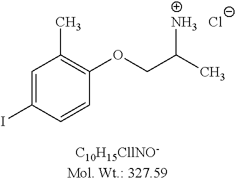 | JME-207 |
| 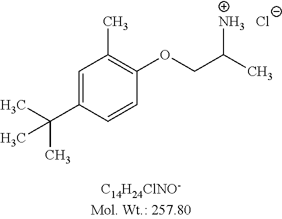 [SMILES: Cc1cc(C(C)(C)C)ccc1OCC(C)[NH3+] ; [Cl-]] | JME-208 |
| 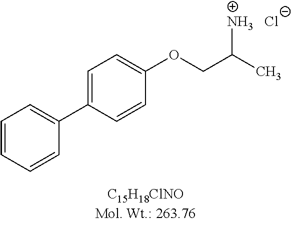 [SMILES: CC([NH3+])COc1ccc(-c2ccccc2)cc1 ; [Cl-]] | JME-209 |
| 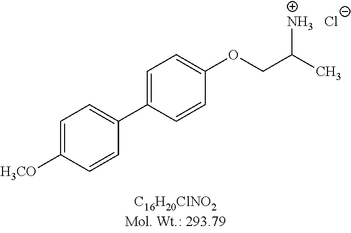 [SMILES: COc1ccc(-c2ccc(OCC(C)[NH3+])cc2)cc1 ; [Cl-]] | JME-209-1 |
| 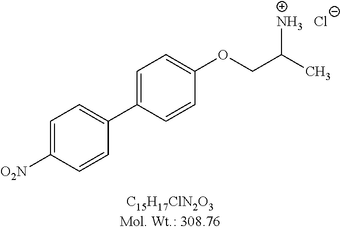 [SMILES: CC([NH3+])COc1ccc(-c2ccc([N+](=O)[O-])cc2)cc1 ; [Cl-]] | JME-209-2 |
| 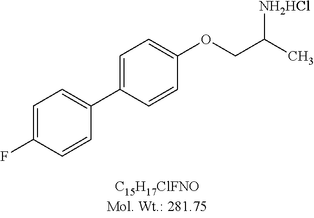 | JME-209-3 |
| 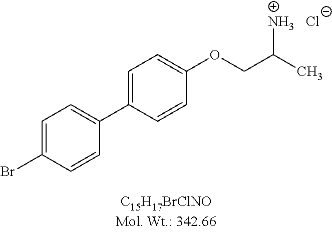 [SMILES: CC([NH3+])COc1ccc(-c2ccc(Br)cc2)cc1 ; [Cl-]] | JME-209-4 |
| 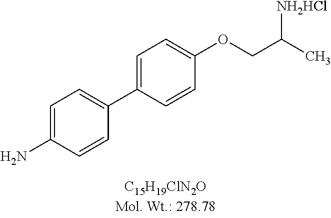 | JME-209-5 |
| 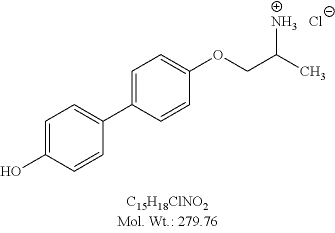 [SMILES: CC([NH3+])COc1ccc(-c2ccc(O)cc2)cc1 ; [Cl-]] | JME-209-6 |
| 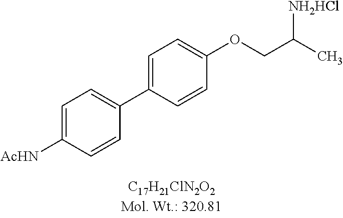 | JME-209-7 |
| 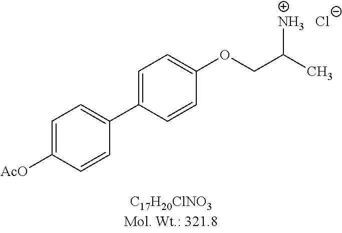 [SMILES: CC(=O)Oc1ccc(-c2ccc(OCC(C)[NH3+])cc2)cc1 ; [Cl-]] | JME-209-8 |

Examples contemplating the synthesis of the compounds structurally analogous to mexiletine, according to the present invention, will be shown below.

The examples shown herein are intended only to exemplify, but without limiting the scope of the invention.

### Example 1

### Synthesis of Diphenyloxyalkylamine Derivatives of Structural Formula II

The starting material substituted phenyl-phenol (29.38 mmol) was dissolved in acetone along with sodium carbonate (1-5 eq.) and catalytic amount of potassium iodide. After previous reflux, a solution of chloroacetone (1-3 eq.) was added over 0.5 to 2 hours, remaining under reflux for 2 to 5 hours. The medium was evaporated to dryness, to follow with the addition of water (30 mL) and extraction with ethyl acetate. The organic phase was dried and evaporated to give the first intermediate in the form of a dark oil (70-90%).

The propanone, obtained as above (29.32 mmol), was dissolved in methanol and the medium cooled in ice bath, to follow with the addition of excess sodium borohydride (2-5 eq.). The reaction medium was stirred at room temperature for 2-5 hours. After addition of water, the reaction medium remained under stirring for another 30-60 minutes. The medium was concentrated at reduced pressure and extracted with ethyl acetate. A colorless oil was obtained after drying and evaporating the organic phase.

The above oil was dissolved in pyridine (10-30 mL) and the medium cooled in ice bath. Excess tosyl chloride (1 to 5 eq.) was added for up to 15 minutes. After 12-24 hours of reaction, the medium was added a solution of HCl until reaching pH 2 to 5. After stirring at room temperature, a white solid was precipitated in the reaction medium, which was removed by filtration. After drying, the solid was dissolved in methanol and reaction was performed with sodium azide (3 eq.) under reflux for 2-6 hours. After evaporation of the solvent, water was added and extracted with ethyl acetate. The organic phase was dried and evaporated to obtain the second intermediate, in the form of a yellowish oil (50-70%).

The azide, obtained as above (16.6 mmol), was dissolved in methanol and catalyst Pd/C was added to this solution. The medium was bubbled with H₂ for up to 10 minutes and then allowed to stir in the presence of this gas for 2-10 hours. After filtering the palladium, the filtrate was evaporated to obtain an oil which was subsequently dissolved in acetone and filtered again. This solution was cooled in ice bath and treated with HCl gas flow until reaching pH 1-5. The precipitate was isolated by filtration followed by washing with cold acetone. The final product was obtained after drying, as a white solid, which spectral data are listed below:

JME 209 [obtained from 4-phenyl-phenol according to the synthesis described in the Example 1]: M.P.: 252-254° C.; ¹H NMR (MeOD, 500 MHz) 7.1-7.6 (m, 9H, ArH), (4.0-4.2, m, 2H, —O—CH₂—), 3.75 (m, 1H, —CH—), 1.44 (d, 3H, CH₃), ¹³C NMR (MeOD, 125 MHz): 159.13, 141.97, 136.18, 130.60, 129.94, 129.34, 128.68, 128.38, 127.39, 127.10, 116.83, 115.55, 69.98, 47.87, 15.11; IR (KBr): 4368, 3047, 1924, 1608, 1049, 883, 812, 437; GC-MS (100%): m/z 227 (free base).

JME 257 [obtained from 4-(4′-Bromo-phenyl)-phenol according to the synthesis described in the Example 1] M.P.:>300° C.; ¹H NMR (MeOD, 400 MHz) 7.1-7.6 (m, 8H, ArH), 4.0-4.3 (m, 2H, —O—CH₂—), 3.75 (m, 1H, —CH—), 1.44 (d, 3H, CH₂, J=6.76 Hz); ¹³C NMR (MeOD, 100 MHz): 159.45, 141.06, 134.86, 130.60, 133.07, 129.55, 129.26, 122.01, 116.36, 70.17, 48.56, 15.59; IR (KBr): 3000, 2989, 1603, 1485, 1247, 1041, 810, 734; GC-MS (99%): m/z 305 (free base).

JME 260 [obtained from 4-(4′-fluoro-phenyl)-phenol according to the synthesis described in the Example 1] M.P.: 225-227° C.; ¹H NMR (MeOD, 400 MHz): 7.0-7.6 (m, 8H, ArH), (4.0-4.3, m, 2H, —O—CH₂—), 3.75 (m, 1H, —CH—), 1.45 (d, 3H, CH₃, J=6.80 HZ); ¹³C NMR (MeOD, 100 MHz): 164.94, 162.51, 159.14, 138.31, 135.16, 129.54, 129.46, 129.25, 116.69, 116.48, 116.30, 70.16, 48.56, 15.61; IR (KBr): 2968, 2879, 1597, 1498, 1230, 1041, 815, 559, 513; GC-MS (100%): m/z 245 (free base).

### Example 2

### Synthesis of Aryloxyalkylamine Derivatives of Structural Formula III

The appropriately substituted phenol derivative (24.88 mmol) was dissolved in acetone along with potassium carbonate (1 to 5 eq.) and catalytic amount of potassium iodide. Under reflux, a solution of chloroacetone (1 to 3 eq.) was added in acetone for a period of 0.5-2 hours, remaining in this condition for a further period of 2-5 hours. Then, water was added and extracted with ethyl acetate. The organic phase was dried and evaporated to give the first intermediate in the form of a dark oil (70-95%).

The propanone, obtained as above (24.51 mmol), was dissolved in methanol and the medium cooled in ice bath, to follow with the addition of excess sodium borohydride (2-5 eq.). The reaction medium was stirred at room temperature for 2-5 hours. After addition of water, the reaction medium remained under stirring for another 30-60 minutes. The medium was concentrated at reduced pressure and extracted with ethyl acetate. A colorless oil was obtained after drying and evaporating the organic phase.

The above oil was dissolved in pyridine (20-50 mL) and the solution formed was cooled in ice bath. Excess tosyl chloride (1 to 5 eq.) was added for up to 15 minutes. After 12-24 hours of reaction, the medium was added a solution of HCl until reaching pH 2 to 5. After stirring at room temperature, a white solid was precipitated in the reaction medium, which was removed by filtration. After drying, the solid was dissolved in methanol and reaction was performed with sodium azide (3-7 eq.) under reflux for up to 20 hours. After evaporation of the solvent, water was added and extracted with ethyl acetate. The organic phase was dried and evaporated to obtain the second intermediate, in the form of a yellowish oil (50-70%).

The azide, obtained as above (12.32 mmol), was dissolved in tetrahydrofuran and this solution was added triphenylphosphine (1-2 eq). The medium was then stirred at room temperature for up to 20 hours. Then, water was added and the reaction medium was heated until reflux, after which it was maintained for up to 3 hours. The organic solvent was removed by evaporation to form an oil which was dissolved again in acetone (30 mL). After cooling, the solution was subjected to HCl gas flow until pH 2-3, water was added and extracted with ethyl ether. The aqueous phase was basified until pH 10-12 and extracted with ethyl acetate. The organic phase was dried and concentrated to give an oil which was subsequently dissolved in acetone. The solution was cooled in ice bath and subjected to HCl gas flow until the medium pH is between 1 and 5, leading to the formation of a precipitate. The precipitate was isolated by filtration followed by washing with cold acetone. The final product was obtained after drying, as a white solid, which spectral data are listed below:

JME 141 (obtained from 3-iodine-phenol according to the synthesis described in the example 2): M.P.: 204-206° C.; ¹H NMR (D₂O, 500 MHz): 7.0-7.5 (m, 4H, ArH), 4.0-4.3 (m, 2H, —O—CH₂—), 3.8 (m, 1H, —CH—), 1.4 (d, 3H, —CH₃ J=6.5 Hz); ¹³C NMR (MeOD, 125 MHz): 158.17, 131.88, 130.58, 123.84, 114.39, 94.56, 73.06, 55.76, 16.28; IR (KBr): 3109, 2985, 1585, 1502, 1465, 1290, 1008, 763, 682; GC-MS (1000): m/z 277 (free base).

JME 170 (obtained from 2-chloro-5-methyl-phenol according to the synthesis described in this example 2): M.P.:220-222° C.; ¹H NMR (D₂O, 400 MHz): 7.22 (m, 1H, ArH₆,), 7.05 (m, 2H, ArH_{2,4}) 4.0-4.3 (m, 2H, —O—CH₂—), 3.90 (m, 1H, —CH—), 2.27 (s, 3H, ArCH₃), 1.51 (d, 3H, —CH₃, J=6.8 Hz); ¹³C NMR (MeOD, 100 MHz): 156.32, 131.63, 131.33, 125.88, 121.31, 112.37, 68.98, 47.09, 30.26, 14.90, 14.39; IR (KBr): 3028, 1593, 1492, 1246, 1049, 854, 655; MS (ES): 200 (M+H)

JME-173 (obtained from 3,5-dimethyl-4-bromo-phenol according to the synthesis described in the example 2): M.P.:220-222° C.; ¹H NMR (D₂O, 500 MHz): 6.8 (m, 2H, ArH), 4.0-4.2 (m, 2H, —O—CH₂—), 3.80 (m, 1H, —CH—), 2.3 (s, 6H, ArCH₃), 1.4 (d, 3H, —CH₃ J=10 Hz); ¹³C NMR (MeOD, 125 MHz): 156.28, 139.78, 118.54, 114.27, 68.52, 47.00, 23.06, 14.16; IR (KBr): 3066, 2978, 2120, 1739, 1585, 1468, 1319, 1172, 1018, 856, 812, 663; CG-MS (100%): m/z 227 (free base).

JME 207 (obtained from 2-methyl-4-iodide-phenol according to the synthesis described in the example 2): M.P.:233-235° C.; ¹H NMR (MeOD, 500 MHz): 7.4 (m, 3H, ArH), 4.0-4.2 (m, 2H, —O—CH₂—), 3.7 (m, 1H, —CH—), 2.2 (s, 3H, ArCH₃), 1.4 (d, 3H, —CH₃ J=6.8 Hz); ¹³C NMR (MeOD, 125 MHz): 157.59, 139.84, 136.44, 130.71, 114.40, 84.66, 47.79, 15.67 IR (KBr): 3066, 2978, 2120, 1739, 1585, 1468, 1319, 1172, 1018, 856, 812, 663; CG-MS (100%): m/z 291 (free base).

Melting points were determined in 130 fisatom apparatus and are uncorrected. Analyses of Proton Magnetic Resonance (1H NMR) were determined in Bruker AC 400 spectrometer at 400 MHz or 500 MHz. Multiplicities were designated as: s, singlet; d, doublet; t, triplet; dd, double doublet; m, multiplet; bs, broad signal. Analyses of Carbon Magnetic Resonance (13C NMR) were determined at 100 MHz or 125 MHz. Infrared spectra were obtained in a Perkin-Elmer 467 FTIR spectrometer using potash bromide pellets. Mass spectra were obtained in GC/MS column 122 5532 apparatus Agilent by electron impact. The progress of all reactions was monitored by thin layer chromatography, using aluminum chromate films (2.0×6.0 cm, 0.25 mm; silica gel 60, HF-254, Merck) with the aid of ultraviolet light at 264 nm. For purification by chromatography column, silica gel was used (230-400 mesh).

The following examples illustrate the pharmacological properties of the compounds of the present invention in comparison to prototype compound mexiletine. They also illustrate the potential of these analogues on the inhibition of pulmonary inflammatory diseases, such as asthma and COPD.

### Example 3

### Evaluation of the Blocking Potency of the Sodium Current Exhibited by Mexiletine Compared with the Analogues JME-141, JME-173, JME-188, JME-207, JME-209, JME-257 and JME-260

### A. Method and Evaluation

Pituitary GH3 cells obtained from mice were grown in RPMI 1640 medium containing 10% fetal bovine serum, penicillin (100 U/ml) and streptomycin (100 μg/ml). The cells were kept at 37° C. in a humidified atmosphere with 5% CO₂ and grown in slides for 1-2 before use. The ion channel currents in GH3 cells were recorded according to the “Patch Clamp” technique, as previously described (Neper et al., 1992). The slides containing adhered cells were placed in a chamber attached to a microscope, and continuously infused with saline with the following composition (mM): NaCl (150), KCl (5), MgCl₂ (1), CaCl₂ (0.01) EGTA (1), HEPES (10), BaCl₂ (2), and CdCl₂ (0.1). The solution pH was adjusted to 7.4 at room temperature with the aid of a NaOH solution. The cells were observed in inverted microscope in phase contrast mode (Axiovert 100, Carl Zeiss, Oberkochem, Germany). The voltage clamp records in the “whole cell” configuration with gigaohm sealing (>10 GΩ) were obtained using an Axopatch-1D amplifier (Axon instruments, San Mateo, Calif.). Sodium currents were recorded in saline with or without the tested compounds. The series resistance was 6-10 MS) for all experiments, when the pipette was filled with intracellular saline solution with the following composition (mM): KCl (150), NaCl (5), MgCl₂ (1), HEPES (10), and EGTA (0.1). The saline solution pH was adjusted to 7.4 at room temperature with the aid of a NaOH solution. Fifteen minutes after the rupture of the membrane patch, the records of the ionic currents were started. The pulse protocols and data acquisition were controlled by an interface (Axon Instruments, Palo Alto, Calif.) and acquired using Clampex 9 software. The records of sodium currents were filtered at 1 kHz and sampled at 8 kHz. Around 26% of series resistance was compensated electronically. The drugs were applied to the chamber by gravity. The infusion rate was maintained at 0.8 to 1.1 ml/minute and the bath volume was approximately 50 μl.

### B. Statistical Analysis

The results were expressed as mean±Standard Error of the Mean. Statistical differences were determined by using tests of analysis of variance, followed by the Student-Newman-Keuls test. p-Values lower than or equal to 0.05 were considered significant.

### C. Results

Table 1 shows the concentrations of substances capable of inhibiting by 50% (IC₅₀) the sodium current in the target cells. By the patch clamp technique, it was found that the depolarization (−90 mV to 60 mV) of rat pituitary cells (GH3 cell line) generated sodium currents that were inhibited, on a concentration-dependent form by pre-treatment with tetrodotoxin (IC₅₀=304 nM) (data not shown), while the prototype substance mexiletine showed an IC₅₀ for blocking the sodium current of the order of 278 μM. Table 1 also shows that the analogous compounds showed IC₅₀ values between 178 and 1208 times higher than that shown by co-incubation with mexiletine.

TABLE 1
Comparative Effect of inhibition of sodium current evidenced
by mexiletine and analogues of the invention, from the
classes of diphenyloxyalkylamines and aryloxyalkylamines,
in GH3 cells evaluated in the patch clamp system.

| Compounds | IC₅₀ (mM) | N |
|---|---|---|
| Mexiletine | 0.278 | 4 |
| JME-141 | 222 | 3 |
| JME-207 | 177 | 4 |
| JME-173 | 183 | 4 |
| JME-188 | 199 | 4 |
| JME-209 | 49 | 4 |
| JME-257 | 336 | 4 |
| JME-260 | 107 | 4 |

Thus, the ranking of blocking potency of the sodium current in this system would be mexiletine>>>JME-209>JME-260>JME-207>JME-173>JME-188>JME-141>JME-257.

Since the main undesirable effects of mexiletine (including cardiovascular depression) result directly from the suppressing activity of sodium currents, it is possible to hypothesize that the analogous compounds have a lower toxicity potential compared to the prototype mexiletine.

### Example 4

### Inhibitory Activity of the Contraction of Tracheal Smooth Muscle of Rat Presented by the Compounds Mexiletine, JME-173 and JME-207

### A. Method and Evaluation

All experimental procedures involving animals regarding this patent application were approved by the Ethics Committee on Animal Use of Oswaldo Cruz Foundation (CEUA License—LW-23/10).

In this study, Wistar rats of both sexes were used, weighing between 200 and 250 g, coming from the Laboratory Animal Breeding Center of Fundacdo Oswaldo Cruz. As previously described (Coelho et al., 2008), the animals were sacrificed by exposure to air atmosphere enriched with CO₂, Then, the anterior cervical region was opened so that the trachea could be located and removed. It was then transferred to a Petri dish containing Krebs solution with the following composition (mM): NaCl (118), KCl (4.8), CaCl₂ (2.5), MgSO₄ (1.2), KH₂PO₄ (1.2), NaHCO₃ (24), and glucose (11). The total segment was divided into fragments of about 3 to 4 rings, which were kept in another Petri dish containing Krebs solution. Each fragment was mounted vertically on a 10 ml cuvette with Krebs solution maintained at 37° C. and aerated with carbogen mixture (95% O₂ and 5% CO₂). The lower rod is fixed to the cuvette base and the top portion was attached to the isometric transducer for measuring the voltage variation of the fragment. The transducer was connected to a device which transforms the voltage variation in digital record. The fragments were subjected to a basal voltage of 1 g and were calibrated, so that the subsequent contractions could be expressed as a percentage of this 1 g voltage. The solutions were introduced inside the cuvettes with the aid of an automatic pipette. The end of the tip was always placed at the same height and position, without touching the muscle. The tracheal rings were initially contracted with 2.5 μM of carbachol. When the contractions reached the plateau, each segment was washed until the total relaxation of the smooth muscle. The compounds mexiletine (30-1000 μM) JME-173 (30-100 μM) and JME-207 (10-100 μM) were added 10 minutes before the addition of increasing concentrations of carbachol (10⁻⁸-10⁻⁴ M). All results were expressed as percentage of the contraction produced by 2.5 μM of carbachol (Coelho et al., 2008).

### B. Statistical Analysis

The values of the mean±Standard Error of the Mean of the groups investigated were statistically analyzed using the test of analysis of variance (ANOVA), followed by Student-Newman-Keuls test. p-Values lower than or equal to 0.05 were considered significant.

### C. Results

Table 2 shows that the compounds JME-173 and JME-207 were equieffective in blocking the contractile response of rat tracheal rings induced by the muscarinic agonist carbachol (10 μM), with IC₅₀ values of 44.4 and 40.9 μM, respectively. Pre-treatment for 10 minutes with JME-173 and JME-207 (100 μM) reached levels of inhibition of the muscarinic contraction of the order of 98% and 93%, respectively. The analogues were about 10 times more potent than the prototype mexiletine, which under the same conditions inhibited 50% of the response (IC₅₀) at the concentration of 466.4 μM, reaching the blocking of about 88% of the muscarinic contraction at the concentration of 1000 μM.

TABLE 2
Potency values (IC₅₀) and maximum effect (EMAX) of inhibition
of the response of (10 μM) carbachol-induced contraction on
rat tracheal rings pretreated for 10 minutes with mexiletine,
JME-173 or JME-207, representatives of the class of aryloxyalkylamines.
Data represent the mean ± SEM from 4 to 7 tracheal rings.

| Compounds | IC₅₀ (μM) | EMAX |
|---|---|---|
| Mexiletine | 466.4 | 87.7 ± 4.9 |
| JME-173 | 44.3 | 97.9 ± 4.7 |
| JME-207 | 40.9 | 92.9 ± 4.3 |

### Example 5

### Effect of Mexiletine, JME-173 and JME-207 in the Relaxation of Trachea Pre-Contracted by Carbachol

### A. Method and Evaluation

To assess the potential relaxing effect of the respiratory smooth muscle, rat tracheas were obtained and maintained in an isolated organ bath, as previously described (Coelho et al., 2008). The tracheal segments were then pre-contracted with carbachol at the concentration of 2.5 μM and subjected to increasing concentrations of the tested compounds. All results were expressed as percentage of the contraction produced by carbachol (Coelho et al., 2008).

### B. Statistical Analysis

The values of the mean±Standard Error of the Mean of the groups investigated were statistically analyzed using the test of analysis of variance (ANOVA), followed by Student-Newman-Keuls test. p-Values lower than or equal to 0.05 were considered significant.

### C. Results

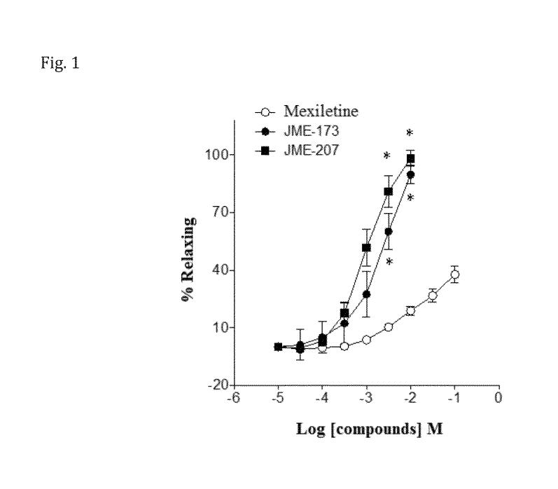

FIG. 1 shows the relaxing effect of the compounds JME-173 and JME-207 in comparison to the prototype compound mexiletine. It was observed that at conditions of pre-contraction by carbachol, the addition of mexiletine (10 μM-100 mM), in isolated tracheal ring system, caused a relaxation which increased with increasing concentration of the prototype mexiletine, to achieve a maximum relaxation effect of 40%±3% (n=10). On the other hand, treatments with JME-173 and JME-207 (10 μM-10 mM) resulted in maximum relaxation of 118%±21% (N=11) (mean±SEM) and 111%±8% (N=9), respectively. Under these conditions, the EC₅₀ values of relaxation for mexiletine, JME-173 and JME-207 were 146.4 mM±39.2 mM (mean±SEM; N=10), 3.1 mM±0.8 mM (N=9) and 1.0 mM±0.3 mM (N=8), respectively. The findings show that the relaxing potency of the analogues is significantly higher than that evidenced by the prototype in this preparation. More specifically, the compounds JME-173 and JME-207 were, in this order, 47 and 146 times more potent as relaxation inducers than mexiletine. The results reinforce the interpretation that these analogues have therapeutic application in the control of spasm of airways.

### Example 6

### Antispasmodic Activity of the Compound JME-173 Evaluated in the System of Anaphylactic Contraction of Tracheal Ring

### A. Method and Evaluation

In this study, Wistar rats of both sexes were used, weighing between 200 and 250 g, coming from the Laboratory Animal Breeding Center of Fundacdo Oswaldo Cruz. As described previously (da Costa et al., 2007), the animals were sensitized by injection into the dorsal subcutaneous tissue with mixture containing 50 μg of ovalbumin and 5 mg of aluminum hydroxide on days 0 and 7. On the 14^{th} day after the first sensitization, the animals were sacrificed for the removal of the trachea. After a stabilization period of 30 minutes, the tracheal rings were contracted initially with carbachol (2.5 μM) for testing the feasibility and reproducibility of the responses of the preparation.

### Treatment and Anaphylaxis Challenge

The tracheal rings were exposed to increasing concentrations of JME-173 (3-30 μM), or carrier (0.9% NaCl) for 30 minutes before the triggering of the contractile response triggered by ovalbumin (100 μg/ml). Salmeterol (30 μM) was used as reference treatment. The responses were expressed as mean±Standard Error of the Mean of at least 5 tracheal segments. All results were expressed as percentage of the contraction produced by 2.5 μM of carbachol.

### B. Statistical Analysis

The values of the mean±Standard Error of the Mean of the groups investigated were statistically analyzed using the test of analysis of variance (ANOVA), followed by Student-Newman-Keuls test. The statistical evaluation of the data obtained for treatment with salmeterol was performed using the Student's T-test. p-Values lower than or equal to 0.05 were considered significant.

### C. Results

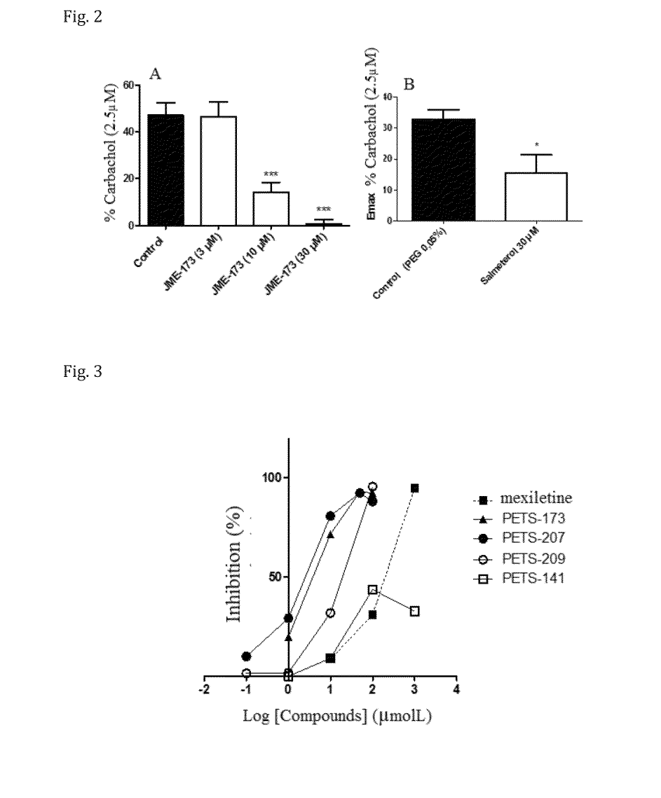

FIG. 2A shows the antispasmodic effect, concentration-dependent, of the compound JME-173 on the contractile response induced by the addition of the allergen. It was observed that JME-173 inhibited 50% of the anaphylactic contraction response (EC₅₀) at the concentration of 8.3 μM. The response was completely inhibited after treatment with 30 μM of JME-173. Importantly, at the same concentration (30 μM), the blocking exhibited by salmeterol was only 52% (FIG. 2B). The findings demonstrate that JME-173 was more potent than salmeterol in blocking anaphylactic contraction. It was also evident the greater antispasmodic activity of JME-173 in the anaphylactic contraction system, in comparison to the blocking exhibited by this compound on carbachol-induced contraction.

### Example 7

### Anti-Anaphylactic Activity of the Compounds Mexiletine, JME-173, JME-207 and JME-209, Evaluated in the System of Degranulation of Sensitized Mast Cells Induced by Antigen

### A. Method and Evaluation

For this study, mast cells of the RBL-2H3 line were used, as previously reported (Beaven et al., 1987). The cells were maintained in D-MEM medium supplemented with 15% fetal bovine serum, penicillin (100 IU/ml) and streptomycin (0.1 mg/ml), and placed in an oven at 37° C. and atmosphere of 5% CO₂ until reaching confluence. The cells were then dissociated from the plate using trypsin, centrifuged at 1000 rpm for 5 minutes and distributed in 48-well plates at a density of 125,000 cells per well. The cells were sensitized with monoclonal DNP-specific IgM (1 μg/mL) diluted in the same medium used for the cultivation and maintained in the oven for 20 hours. After this period, the cells were washed with Tyrode-gelatin and subjected to treatment with increasing concentrations of JME-173, JME-207, JME-209 or mexiletine for 60 minutes. Then, incubation was performed with DNP-BSA (10 ng/ml) for a further period of 60 minutes. After this period, 10 μL of supernatant were collected from each well and added to a 96-well plate. The cells were lysed with 200 μL of 0.1% Triton X-100 and 10 μL of the lysate of each plate were added to the 96-well plate. Then, 40 μL of substrate for the β-hexosaminidase enzyme were added to the samples. After 40 minutes of reaction, reaction stopping solution (0.2 M glycine) was added, generating a colorimetric response, which was measured by spectrophotometer (λ=405 nm).

The compounds JME-173, JME-207, JME-209 and mexiletine were also evaluated for their cytotoxic potential, based on the Alamar Blue assay, as previously reported (Czekanska, 2011). In this test, the compound terfenadine was used as positive control.

### B. Statistical Analysis

The results were expressed as inhibition percentage. Statistical differences were determined by using tests of analysis of variance, followed by the Student-Newman-Keuls test. p-Values lower than or equal to 0.05 were considered significant.

### C. Results

Several local anesthetic agents, such as lidocaine, inhibit mast cell degranulation induced by mechanisms mediated or not mediated by IgE, by blocking calcium channels (Yanagi et al., 1996). Our results showed that the compound mexiletine also inhibited the anaphylactic degranulation of mast cells at concentrations ranging from 100 μM to 1000 μM (FIG. 3). The same figure shows that the analogues studied JME-173, JME-207 and JME-209 were equally effective in blocking mast cell degranulation caused by exposure to the allergen, evidencing, however, greater potency of the analogues studied when compared to mexiletine.

Table 3 shows the comparative potency values (IC₅₀) and efficacy (EMAX) of the compounds studied. All of them inhibited by about 100% the degranulation response, whereas IC₅₀ values decreased from 381.8 μM, obtained after treatment with mexiletine, to 28.6 μM, 3.4 μM and 2.3 μM after JME-209, JME-173 and JME-207, respectively.

These results, obtained with mast cells passively sensitized with IgE, indicate that the analogues JME-173, JME-207 and JME-209 were capable of inhibiting the anaphylactic activation of mast cells with higher potency (up to two orders of magnitude) when compared to the prototype.

TABLE 3
Comparative values of inhibition potency (IC50) and maximum effect
(EMAX) of mexiletine, JME-207, JME-173 and JME-209, of the classes
of diphenyloxyalkylamines and aryloxyalkylamines, relative to
the blocking of anaphylactic mast cell degranulation.

| Compounds | IC₅₀ (μM) | EMAX |
|---|---|---|
| Mexiletine | 381.8 | 100 |
| JME-207 | 2.3 | 94 |
| JME-173 | 239.4 | 97 |
| JME-209 | 28.6 | 100 |

### Example 8

### Bronchodilator Activity of the Compounds JME-207 and JME-209 In Vivo

### A. Method and Evaluation

A/J mice of both sexes were used, weighing between 18 and 20 g, coming from the Laboratory Animal Breeding Center of Oswaldo Cruz Foundation. Using barometric whole-body plethysmography (Buxco Research System, Wilmington, N.C.), bronchospasm responses caused by subsequent inhalations of methacholine (12, 25 and 50 mg/ml for 2.5 minutes, 5-minute intervals) were measured in standard A/J mice, awake, not immobilized, as previously reported (Coelho et al., 2008; Hamelmann et al., 1997). Penh measures in response to methacholine challenge were performed 1 hour and 3 hours after treatment with JME-207 and JME-209 (30 and 100 mg/kg) administered orally (gavage).

### B. Statistical Analysis

The results were expressed as mean±Standard Error of the Mean. Statistical differences were determined by using tests of analysis of variance, followed by the Student-Newman-Keuls test. p-Values lower than or equal to 0.05 were considered significant.

### C. Results

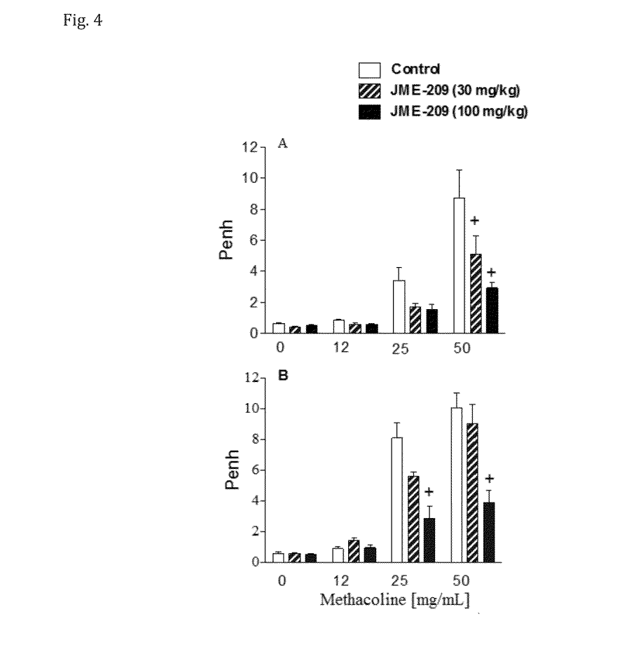

FIG. 4 shows the effect of treatment with JME-209 (30 and 100 mg/kg, orally) or carrier (0.9% NaCl) on the response of increase of Penh (indicative of increase in lung resistance) induced by methacholine challenge (12-50 mg/ml) in the times 1 hour and 3 hours after treatment. There was a slight blocking of cholinergic bronchospasm with both doses used (30 and 100 mg/kg) in the analysis conducted 1 hour after treatment. The blocking shown to be active only at the highest dose, when the tests of stimulation response with methacholine was repeated 3 hours after treatment, suggesting that the compound has an action time of at least 3 hours when the substance is administered orally at a dose of 100 mg/kg.

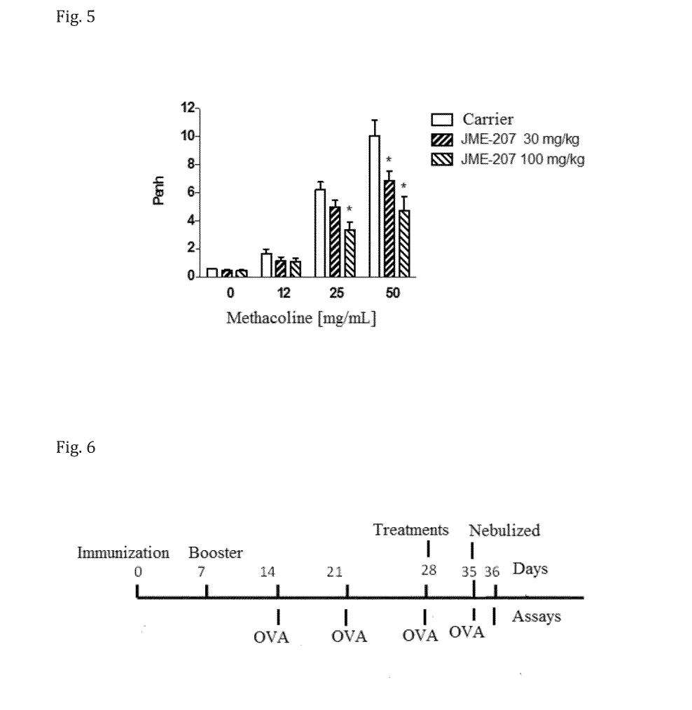

Similar results were obtained when the animals were treated with JME-207. Administered orally, at doses of 30 and 100 mg/kg, the compound significantly inhibited the response of methacholine-induced bronchoconstriction 3 hours after treatment (FIG. 5). The results together demonstrate the antispasmodic activity of the compounds JME-209 and JME-207, in vivo, confirming our data obtained in isolated organ system (in vitro).

### Example 9

### Therapeutic Effect of the Compounds JME-141, JME-173, JME-207 and JME-188 on Lung Inflammation and Hyperreactivity in Asthma Model in Mice

### A. Method and Evaluation

Male A/J mice (18-20 g), coming from the Laboratory Animal Breeding Center of Oswaldo Cruz Foundation, were used in the experiments. The sensitizing and antigen challenge procedures used in this study followed the experimental protocol shown in FIG. 6. The animals were previously sensitized with a mixture of ovalbumin (OVA) (50 μg) (Grade V; Sigma, St. Louis, Mo., USA) and aluminum hydroxide (5 mg) administered subcutaneously on day 0 with boost on day 14 (equal suspension administered intraperitoneally). Nasal instillations of OVA (25 μg/25 μl in sterile 0.9% NaCl) were administered on days 14, 21, 28 and 35, with the hyperresponsiveness analysis performed 24 hours after the last challenge. As shown in FIG. 6, the treatments with JME-141, JME-173, JME-207, and JME-188 were carried out only on days 28 and 35, 1 hour before the challenge with OVA, by nebulization for 30 minutes. That is, the treatments took place after the installation of asthma framework, reflecting a therapeutic action of the respective compounds analyzed.

The effect of the treatments on bronchial hyperreactivity was investigated by measuring the resistance changes and pulmonary elastance, using invasive barometric plethysmography whole-body system (Buxco, USA), as previously described (Olsen et al., 2012).

### B. Statistical Analysis

The results were expressed as mean±Standard Error of the Mean. Statistical differences were determined by using tests of analysis of variance, followed by the Student-Newman-Keuls test. p-Values lower than or equal to 0.05 were considered significant.

### C. Results

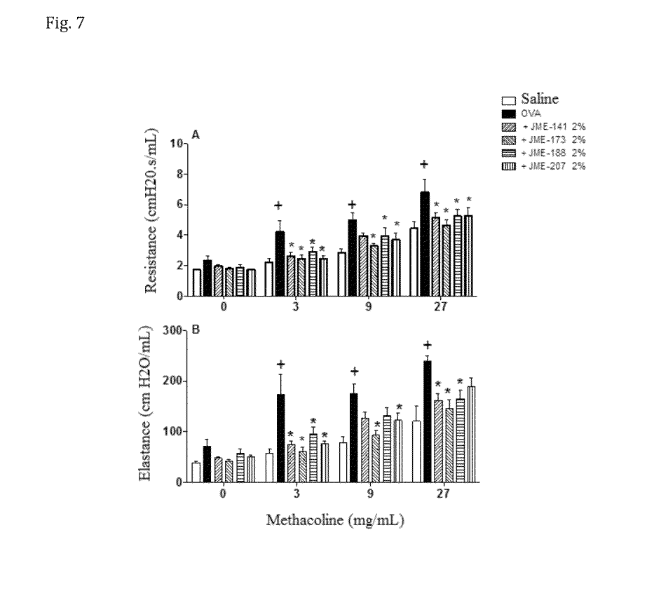

As shown in FIG. 7, the nebulization of the animals for 30 minutes with the compounds JME-141, JME-173, JME-207 and JME-188 (2%), starting from the third week of allergen challenge, abolished the framework of hyperreactivity of airways, observed in animals antigenically sensitized and challenged, treated only with carrier (Tween-80, 0.2%). The inhibition was found to be effective both in lung resistance increasing response, as in the increase of lung elastance observed after exposure of methacholine.

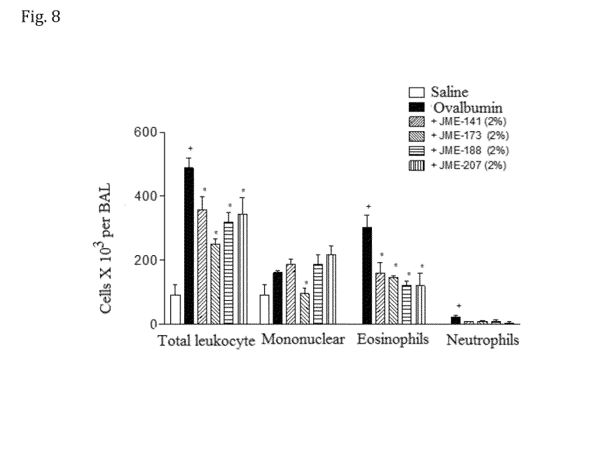

All compounds were equally active in blocking leukocyte infiltration, evaluated by bronchoalveolar lavage, especially for the inhibition of eosinophilic infiltration, inhibited by 50% by all compounds tested (FIG. 8). Analyses of cellularity were also carried out 24 hours after the last antigen challenge.

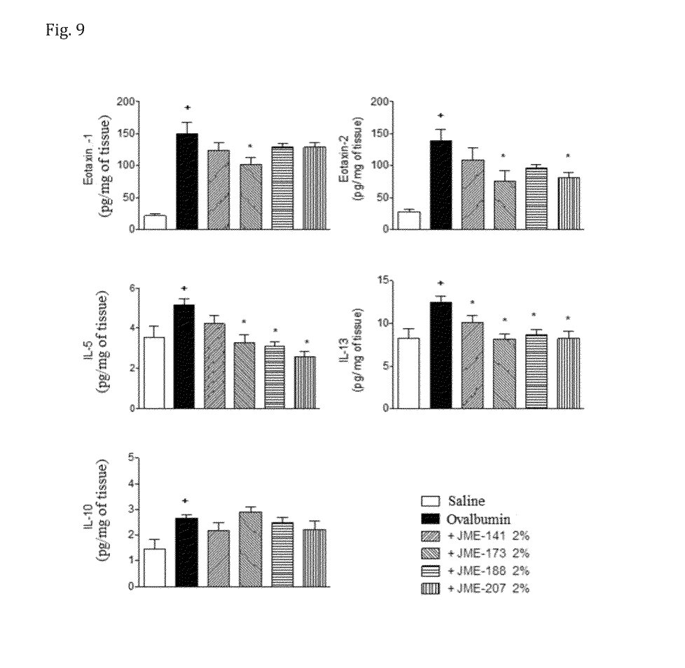

Our results indicated that the inhibition of hyperresponsiveness and cellular recruitment responses, as evidenced by treatment with JME-173, was shown to be associated to the blocking of pro-inflammatory cytokine generation including eotaxin-2, IL-5 and IL-13, without change in the increased levels of the anti-inflammatory cytokine IL-10 (FIG. 9).

The production of cytokines IL-5 and IL-13 was equally sensitive to the treatment with JME-207 or JME-188, but only the latter inhibited eotaxin-2, while both failed to modify the increased production of eotaxin-1. These data indicate that, with minor particularities, the compounds JME-141, JME-173, JME-207 and JME-188, administered via nebulization (2%), are active in blocking lung inflammation and hyperreactivity associated with the asthmatic response. The joint results also suggest that the inhibition of the generation of the pro-inflammatory Th₂ cytokines can be involved in the blocking of pathological features of asthma profile observed in this model.

### Example 10

### Effect of Nebulization with TME-173 on Inflammation, Mucus Production and Lung Remodeling in Murine Model of Asthma

### A. Method and Evaluation

Male A/J mice (18-20 g), coming from the Laboratory Animal Breeding Center of Oswaldo Cruz Foundation, were used in the experiments. The sensitization procedures, antigen challenge and treatment used in this study followed the experimental protocol shown in FIG. 6. Histological techniques were used for the quantification of mucus and peribronchial fibrosis, following previously reported and validated experimental protocols (Serra et al., 2012).

### B. Statistical Analysis

The results were expressed as mean±Standard Error of the Mean. Statistical differences were determined by using tests of analysis of variance, followed by the Student-Newman-Keuls test. p-Values lower than or equal to 0.05 were considered significant.

### C. Results

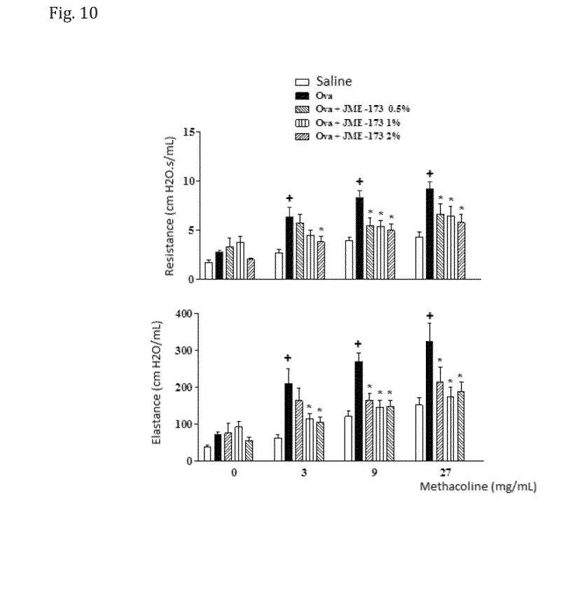

The treatment by nebulization with JME-173 in the concentrations of 0.5%, 1% or 2% for 30 minutes, started at the third week of allergen challenge (FIG. 10), confirmed the antiasthmatic effect of this compound.

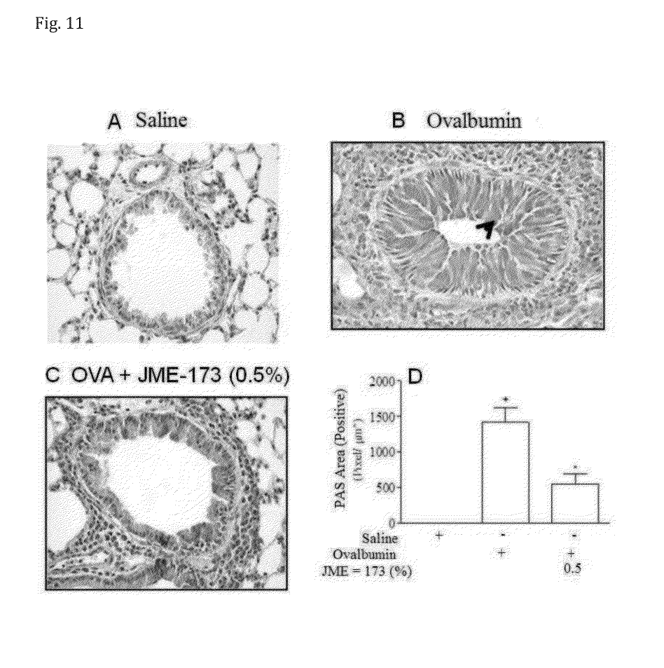

It was evident in the three aerosol concentrations tested that the compound JME-173 was able to block the response of airway hyperreactivity even at the lowest concentration (0.5%), as illustrated in FIG. 11. In this model, challenge with ovalbumin caused a significant increase in the amount of mucus present in the airways of the sensitized animals (micrograph B of FIG. 11) (PAS staining, arrowheads), when compared with control animals challenged with 0.9% saline (micrography B). It is noted in FIG. 11, part C, that the exacerbated mucus production observed in asthmatic mice was substantially inhibited by the treatment with JME-173 (0.5%). FIG. 11D shows the result of quantitative analysis, where it is evident that JME-173 inhibited mucus production by about 70%.

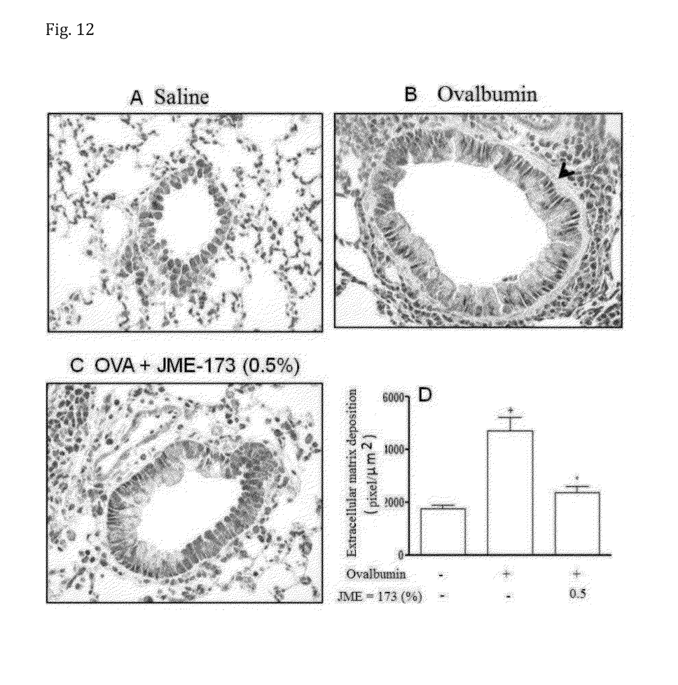

The staining of histological sections of lung tissue with Gömöri trichrome stain evidenced a marked accumulation of extracellular matrix in the peribronchial region (indicated by the arrowhead) in the animals challenged with ovalbumin (FIG. 12, part B), when compared to animals in the negative control group (Part A). The treatment with JME-173 clearly abolished the fibrotic response, as illustrated by the representative image, as well as by the quantitative analysis conducted based on the morphometry (FIG. 12 Part C and D, respectively). Taken together, the data suggest that the nebulization treatment with JME-173 is capable of reversing airways hyperreactivity, as well as inhibits mucus production in the lower airways and peribronchial fibrosis caused by intranasal instillation of allergen agent in sensitized mice.

### Example 11

### Effect of the Compound JME-209 on Lung Inflammation and Airways Hyperreactivity Caused by LPS

### A. Method and Evaluation

Male A/J mice (18-20 g), coming from the Laboratory Animal Breeding Center of Oswaldo Cruz Foundation, were used in the experiments. The mice were anesthetized with halothane aerosol (Cristália, SP, Brazil) to receive intranasal administration of LPS (25 μg/25 μl 0.9% NaCl, instillation) or 0.9% NaCl (25 μl) (negative control). The animals were pretreated with JME-209 (30 and 100 mg/kg, orally) 1 hour before instillation of LPS, and analysis of the impact of treatment on leukocyte recruitment in the airspace was performed 18 hour after challenge. Obtaining of bronchoalveolar lavage, as well as the total and differential leukocyte counts carried out in this effluent, were made as previously described (Kummerle et al., 2012). Thus, after 18 hrs of LPS instillation, the mice were sacrificed by terminal anesthesia with thiopental (500 mg/kg). Then, they had the trachea dissected and cannulated. Bronchoalveolar lavage (BAL) was performed by 3 consecutive lavages of 800 μl of PBS containing EDTA (10 mM). The lavages were then subjected to centrifugation (1500 rpm-10 minutes) and the cell “pellet” resuspended in the volume of 0.5 ml of PBS/EDTA solution 10 mM. The total leukocyte count from the lavage was performed in a Neubauer chamber by light microscopy (100× magnification), diluting an aliquot of the cell suspension from the lavage in TPrk liquid (1:40). The differential counting was performed on cytocentrifuged, which were stained with May-Grunwald-Giemsa and assessed using oil immersion objective (1000× magnification) (Kummerle et al., 2012).

The airway hyperreactivity was also assessed 18 hours after LPS, by exposing the animals to increasing concentrations of aerosolized methacholine (3-27 mg/ml) in FinePoint R/C Buxco® system (Buxco Electronics, Sharon, Conn., USA). The mice were anesthetized with Nembutal (60 mg/kg, i.p.) for the tracheostomy procedure and connection of the animal to mechanical ventilation and pneumotachograph of the FinePoint platform. The neuromuscular activity was blocked with pancuronium bromide (1 mg/kg, i.v.) to enable the pulmonary resistance records (cm H20/mL/s) and elastance (cm H20/mL) in each respiratory cycle, as previously reported (Olsen et al., 2011).

### B. Statistical Analysis

The results were expressed as mean±Standard Error of the Mean. Statistical differences were determined by using tests of analysis of variance, followed by the Student-Newman-Keuls test. p-Values lower than or equal to 0.05 were considered significant.

### C. Results

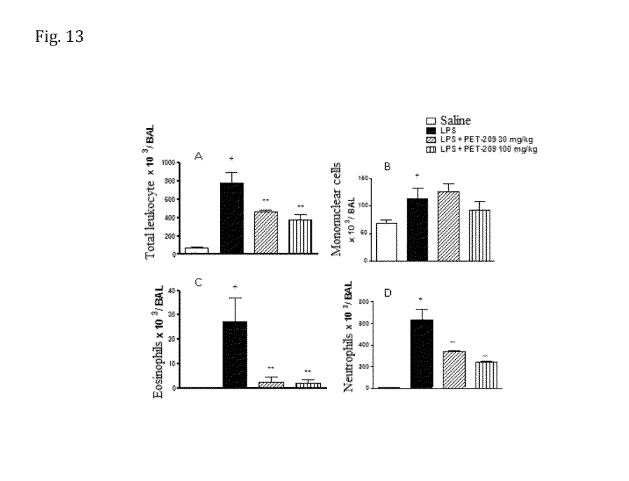

In this model of acute pulmonary inflammation caused by endotoxin, the treatments with JME-209 (30 and 100 mg/kg, orally) administered 1 hour before LPS (25 μg/animal), inhibited leukocyte infiltration in bronchoalveolar space, in particular reducing the levels of eosinophils and neutrophils, without significantly altering the increase in the number of mononuclear cells (FIG. 13).

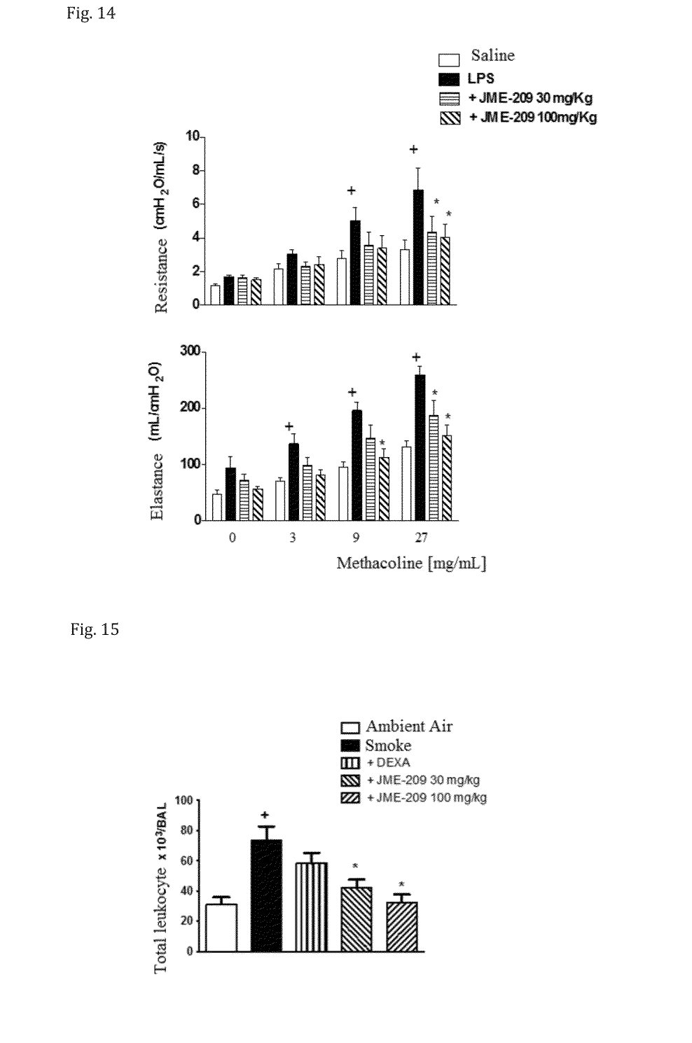

Under these conditions, the treatment also inhibited the mechanical ventilation changes (airway hyperreactivity), represented by the significant increase in lung resistance and elastance values, which are indicators of air flow reduction in the central airways and reduction of expansion capacity of the lung parenchyma, respectively (FIG. 14)

In conclusion, the results show that the pulmonary inflammation and airway hyperreactivity caused by LPS were clearly inhibited by the oral treatment with JME-209, suggesting that this compound has potential for inhibiting chronic pulmonary inflammatory diseases, such as asthma and chronic obstructive pulmonary disease (COPD).

### Example 12

### Protective Effect of TME-209 on Acute Airway Inflammation Induced by Tobacco Smoke in Mice

### A. Method and Evaluation

Male A/J mice (18-20 g), coming from the Laboratory Animal Breeding Center of Oswaldo Cruz Foundation, were used in the experiments. The animals were placed in a chamber and subjected to an atmosphere enriched with 100 ml of smoke from 4 filter cigarettes (trade mark) for 1 minute on four consecutive days. Control animals were exposed to the condition in which cigarette smoke was replaced by equal volume of ambient air (Castro et al., 2009).

The treatments with dexamethasone (1 mg/kg) or JME-209 (30 and 100 mg/kg) were carried out orally 1 hour before each exposure to smoke. The compounds were dissolved in 0.9% NaCl just prior to administration.

Obtaining of bronchoalveolar lavage, as well as the total and differential leukocyte counts carried out in this effluent, were made as previously described (Olsen et al., 2011). Thus, after 24 hrs of the last exposure to cigarette smoke, the mice were sacrificed by terminal anesthesia with thiopental (500 mg/kg). Then, they had the trachea dissected and cannulated BAL was performed by 3 consecutive lavages of 800 μl of PBS containing EDTA (10 mM). The lavages were then subjected to centrifugation (1500 rpm-10 minutes) and the cell “pellet” resuspended in the volume of 0.5 ml of PBS/EDTA solution 10 mM. The total leukocyte count from the lavage was performed in a Neubauer chamber by light microscopy (100× magnification), diluting an aliquot of the cell suspension from the lavage in TPrk liquid (1:40). The differential counting was performed on cytocentrifuged, which were stained with May-Grunwald-Giemsa and assessed using oil immersion objective (1000× magnification).

### B. Statistical Analysis

The results were expressed as mean±Standard Error of the Mean. Statistical differences were determined by using tests of analysis of variance, followed by the Student-Newman-Keuls test. p-Values lower than or equal to 0.05 were considered significant.

### C. Results

In this model of acute pulmonary inflammation by cigarette smoke established in mice (Castro et al., 2009), the treatment with JME-209 (30 and 100 mg/kg, orally) 1 hr prior to challenge with smoke significantly inhibited the accumulation of leukocytes in the bronchoalveolar space, while the treatment with dexamethasone (3 mg/kg, orally) was ineffective (FIG. 15).

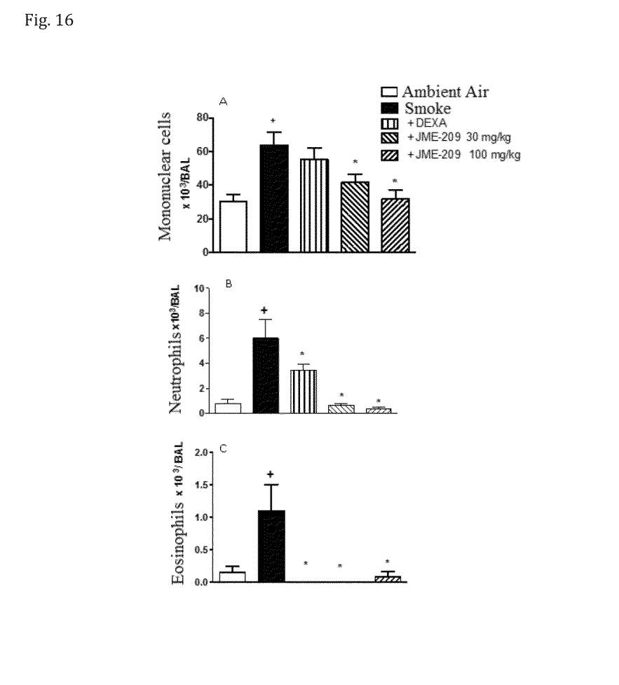

The increase in total leukocytes resulted substantially from increases in the numbers of neutrophils, eosinophils and mononuclear cells in the bronchoalveolar effluent, which changes were blocked by JME-209. The treatment with the steroidal anti-inflammatory Dexamethasone (1 mg/kg, orally) also inhibited the slight increase in the number of eosinophils, but was unable to inhibit the accumulation of mononuclear cells and only partially inhibited neutrophil infiltration (FIG. 16).

In conclusion, considering that cigarette smoke is a major cause of asthma and COPD worsening, the results presented herein strongly suggest that the treatment with JME-209 has the potential to prevent pulmonary inflammation associated with cigarette smoke, an important pathogenesis factor in these patients.

The derivatives of the present invention, as described herein, are usually administered as a pharmaceutical composition. Such compositions may be prepared by procedures well known in the pharmaceutical art and comprise at least one active compound of the invention.

The compounds of this invention are usually administered in a pharmaceutically effective amount. The actual amount of compound administered will be typically determined by a physician, in the light of the relevant circumstances, including the condition to be treated, the chosen route of administration, the compound administered, the age, weight and response of the individual patient, the severity of the symptoms of the patient, and so forth.

The derivatives and compositions described in this patent application can be administered to a subject, preferably a mammal, more preferably a human, to treat and/or prevent the disease by any suitable route.

The compositions containing the derivatives of the present invention may be formulated as:

(1) tablets, capsules, powders for reconstitution;

(2) oral solution;

(3) oral suspension; or

(4) solution for inhalation.

The compositions of the present invention are typically formulated with suitable carriers and may be exemplified as follows.

### (1) Based Formulation for Tablets, Capsules and Powders for Reconstitution

| Component | Pharmaceutically acceptable | |
|---|---|---|
| (function) | carriers | Amount (%) |
| Active | | 10.0-80.0 |
| ingredient | | |
| Disintegrant | Croscarmellose sodium, sodium | 1.0-7.0 |
| | starch glycolate, crospovidone | |
| Glidant | Colloidal silicon dioxide, | 0.5-4.0 |
| | talc | |
| Binder | Polyvinylpyrrolidone (PVP), | 0.5-4.0 |
| | hydroxypropyl methylcellulose | |
| | (HPMC), hydroxypropyl | |
| | cellulose (HPC) | |
| Diluent | Lactose, mannitol, calcium | 5.0-70.0 |
| | phosphate, microcrystalline | |
| | cellulose, pregelatinized | |
| | starch | |
| Flavoring | Strawberry, cherry, orange | 0.05-1.00 |
| Colorant | FD&C red no. 3, FD&C yellow | qs |
| | no. 6 | |
| Sweetener | Aspartame, sodium cyclamate, | 0.05-0.5 |
| | sucralose, saccharin | |
| Lubricant | Magnesium stearate, calcium | 0.5-3.0 |
| | stearate, stearic acid, sodium | |
| | stearyl fumarate | |

### (2) Oral Solution

| Component | Pharmaceutically acceptable | |
|---|---|---|
| (function) | carriers | Amount (%) |
| Active | | 10.0-80.0 |
| ingredient | | |
| Antioxidant | Ascorbic acid, potassium | 0.005-2.0 |
| | metabisulfite, sodium | |
| | metabisulfite, Butylated | |
| | hydroxyanisole (BHA), | |
| | Butylated hydroxytoluene | |
| | (BHT), citric acid | |
| Preservatives | Sodium benzoate, potassium | 0.05-0.5 |
| | benzoate, propylparaben, | |
| | methylparaben, butylparaben, | |
| | potassium sorbate | |
| pH corrector | Citric acid, fumaric acid, | |
| | triethanolamine | |
| Flavoring | Strawberry, cherry, orange | 0.05-1.00 |
| Colorant | FD&C red no. 3, FD&C yellow | 0.01-0.5 |
| | no. 6 | |
| Sweetener | Aspartame, sodium cyclamate, | 0.05-60.0 |
| | sucralose, saccharin, sucrose | |
| Solubilizer | Propylene glycol, cyclodextrin | qs |
| Solvent | Water | qs |

### (3) Oral Suspension

| Component | Pharmaceutically acceptable | |
|---|---|---|
| (function) | carriers | Amount (%) |
| Active | | 10.0-80.0 |
| ingredient | | |
| Suspending | Xanthan gum, sodium | 0.5-5.0 |
| agent | carboxymethyl cellulose, | |
| | methylcellulose | |
| Antioxidant | Ascorbic acid, potassium | 0.005-2.0 |
| | metabisulfite, sodium | |
| | metabisulfite, Butylated | |
| | hydroxyanisole (BHA), | |
| | Butylated hydroxytoluene | |
| | (BHT), citric acid | |
| Preservatives | Sodium benzoate, potassium | 0.05-0.5 |
| | benzoate, propylparaben, | |
| | methylparaben, butylparaben, | |
| | potassium sorbate | |
| pH corrector | Citric acid, fumaric acid, | |
| | triethanolamine | |
| Sweetener | Aspartame, sodium cyclamate, | 0.05-60.0 |
| | sucralose, saccharin, sucrose | |
| Flavoring | Strawberry, cherry, orange | 0.05-1.00 |
| Colorant | FD&C red no. 3, FD&C yellow | qs |
| | no. 6 | |
| Solvent | Water | qs |

### (4) Solution for Inhalation

| Component | Pharmaceutically acceptable | |
|---|---|---|
| (function) | carriers | Amount (%) |
| Active | | 1.0-20.0 |
| ingredient | | |
| Isotonizing | Sodium chloride | 1.0-10.0 |
| agent | | |
| Surfactant | Oleic acid, lecithin, Span 85, | 0.5-5.0 |
| | PVP K25 | |
| pH corrector | Sulfuric acid | qs |
| Solvent | Water | qs |

The carriers (components) described above for the compositions are merely representative. Other materials, as well as processing techniques and the like, are set in specific literature, such as Remington's Pharmaceutical Sciences, 18th edition, 1990, Mack Publishing Company, Easton, Pa., 18042.

Although the present invention has been described with respect to specific embodiments, it is evident that many alternatives and variations are apparent to those skilled in the art. These alternatives and variations should be considered to be supported by the scope of the claims.

Documents belonging to the state of the art of the knowledge of the inventors and cited in the present descriptive report are listed below.
1. U.S. Pat. No. 3,659,019
2. U.S. Pat. No. 3,954,872
3. U.S. Pat. No. 4,031,244
4. Beaven M A, Maeyama K, Wolde-Mussie E, Lo T N, Ali H, Cunha-Melo J R (1987). Mechanism of signal transduction in mast cells and basophils: studies with RBL-2H3 cells. Agents Actions 20(3-4): 137-145.
5. Brightling C E, Gupta S, Gonem S, Siddiqui S (2012). Lung damage and airway remodelling in severe asthma. Clin. Exp. Allergy 42(5): 638-649.
6. Brody H (2012). Chronic obstructive pulmonary disease. Nature 489(7417): S1.
7. Campbell R W (1987). Mexiletine. N. Engl. J. Med. 316(1): 29-34.
8. Castro P, Nasser H, Abrahao A, Dos Reis L C, Rica I, Valenca S S, et al. (2009). Aspirin and indomethacin reduce lung inflammation of mice exposed to cigarette smoke. Biochem. Pharmacol. 77(6): 1029-1039.
9. Coelho L P, Serra M F, Pires A L, Cordeiro R S, Rodrigues e Silva P M, dos Santos M H, et al. (2008). 7-Epiclusianone, a tetraprenylated benzophenone, relaxes airway smooth muscle through activation of the nitric oxide-cGMP pathway. J. Pharmacol. Exp. Ther. 327(1): 206-214.
10. Czekanska E M (2011). Assessment of cell proliferation with resazurin-based fluorescent dye. Methods Mol. Biol. 740: 27-32.
11. da Costa J C, Olsen P C, de Azeredo Siqueira R, de Frias Carvalho V, Serra M F, Alves L A, et al. (2007). JMF2-1, a lidocaine derivative acting on airways spasm and lung allergic inflammation in rats. J. Allergy Clin. Immunol. 119(1): 219-225.
12. Dance A (2012). Health impact: Breathless. Nature 489(7417): S2-3.
13. Groeben H, Foster W M, Brown R H (1996). Intravenous lidocaine and oral mexiletine block reflex bronchoconstriction in asthmatic subjects. Am. J. Respir. Crit. Care Med. 154(4 Pt 1): 885-888.
14. Hamelmann E, Schwarze J, Takeda K, Oshiba A, Larsen G L, Irvin C G, et al. (1997). Noninvasive measurement of airway responsiveness in allergic mice using barometric plethysmography. Am. J. Respir. Crit. Care Med. 156(3 Pt 1): 766-775.
15. Jarvis B, Coukell A J (1998). Mexiletine. A review of its therapeutic use in painful diabetic neuropathy. Drugs 56(4): 691-707.
16. Kummerle A E, Schmitt M, Cardozo S V, Lugnier C, Villa P, Lopes A B, et al. (2012). Design, Synthesis, and Pharmacological Evaluation of N-Acylhydrazones and Novel Conformationally Constrained Compounds as Selective and Potent Orally Active Phosphodiesterase-4 Inhibitors. J. Med. Chem.
17. Marmura M J, Passero F C, Jr., Young W B (2008). Mexiletine for refractory chronic daily headache: a report of nine cases. Headache 48(10): 1506-1510.
18. Monk J P, Brogden R N (1990). Mexiletine. A review of its pharmacodynamic and pharmacokinetic properties, and therapeutic use in the treatment of arrhythmias. Drugs 40(3): 374-411.
19. Neher E, Sakmann B (1992). The patch clamp technique. Sci. Am. 266(3): 44-51.
20. Olsen P C, Coelho L P, da Costa J C, Cordeiro R S, Silva P M, Martins M A (2012). Two for one: Cyclic AMP mediates the anti-inflammatory and anti-spasmodic properties of the non-anesthetic lidocaine analog JMF2-1. Eur. J. Pharmacol. 680: 102-107.
21. Olsen P C, Ferreira T P, Serra M F, Farias-Filho F A, Fonseca B P, Viola J P, et al. (2011). Lidocaine-derivative JMF2-1 prevents ovalbumin-induced airway inflammation by regulating the function and survival of T cells. Clin. Exp. Allergy 41(2): 250-259.
22. Serra M F, Anjos-Valotta E A, Olsen P C, Couto G C, Jurgilas P B, Cotias A C, et al. (2012). Nebulized lidocaine prevents airway inflammation, peribronchial fibrosis, and mucus production in a murine model of asthma. Anesthesiology 117(3): 580-591.
23. Yanagi H, Sankawa H, Saito H, Iikura Y (1996). Effect of lidocaine on histamine release and Ca2+ mobilization from mast cells and basophils. Acta Anaesthesiol. Scand. 40(9): 1138-1144.
24. Remington's Pharmaceutical Sciences, 18th ed., 1990, Mack Publishing Company, Easton, Pa., 18042.

1. A compound which is:
(a) a diphenyloxyalkylamine derivative represented by Formula (II): 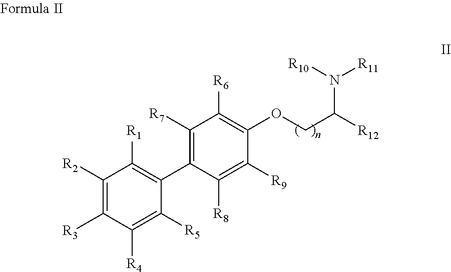
wherein
R₂, R₃, R₄, R₅, R₆, R₇, R₈, R₉ and R₁₂ are independently H, CH₃, OH, CF₃, alkoxide, halogen, linear alkyl radical, branched alkyl radical, cyclic alkyl radical, benzyl group, phenyl, alkene, alkyne, hydroxyalkyl, thioalkyl, acetamide, nitro or an oxygen functionality in an acyclic or cyclic system forming a heterocyclic ring;
the substituents R₁₀ and R₁₁ are independently H, CH₃, linear alkyl radical, branched alkyl radical, cyclic alkyl radical, benzyl group, phenyl, alkene, alkyne, or an oxygen functionality in an acyclic or cyclic system forming a heterocyclic ring; and
n is 1 to 4,
(b) an aryloxyalkylamine derivative represented by Formula (III), 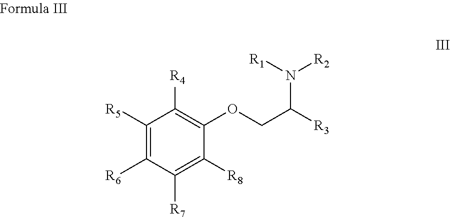
wherein:
R₁ and R₂ are independently H, CH₃, linear alkyl radical, branched alkyl radical, cyclic alkyl radical, benzyl group, phenyl, alkene, alkyne, or an oxygen functionality in an acyclic or cyclic system forming a heterocyclic ring;
R₃ is CH₃, linear alkyl radical, branched alkyl radical, cyclic alkyl radical, benzyl group, phenyl, alkene or alkyne; and
R₄, R₅, R₆, R₇ and R₈ are independently H, CH₃, OH, linear alkyl radical, branched alkyl radical, cyclic alkyl radical, benzyl group, phenyl, alkene, alkyne, ether, thioether, halogen, alkylamine, an electron donor group or an electron remover group, or
(c) an organic salt or a mineral acid salt of the diphenyloxyalkylamine derivative or the aryloxyalkylamine derivative.

2. The compound according to claim 1, which is represented by one of the structures below:

| Structure | Code |
|---|---|
| 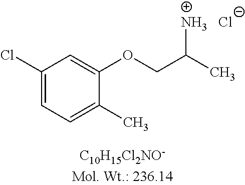 | JME-170 |
| 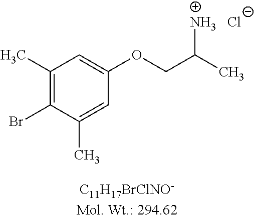 | JME-173 |
| 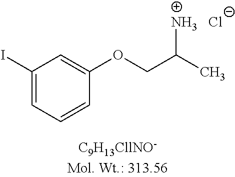 | JME-141 |
| 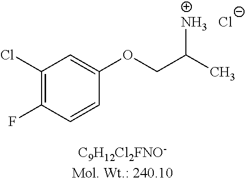 | JME-188 |
| 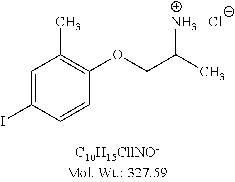 | JME-207 |
| 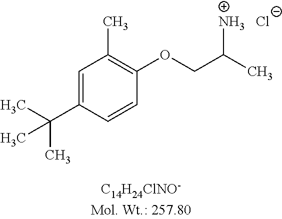 [SMILES: Cc1cc(C(C)(C)C)ccc1OCC(C)[NH3+] ; [Cl-]] | JME-208 |
| 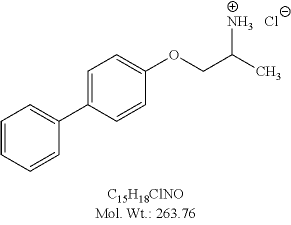 [SMILES: CC([NH3+])COc1ccc(-c2ccccc2)cc1 ; [Cl-]] | JME-209 |
| 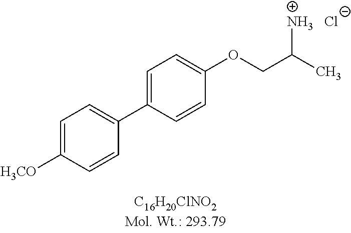 [SMILES: COc1ccc(-c2ccc(OCC(C)[NH3+])cc2)cc1 ; [Cl-]] | JME-209-1 |
| 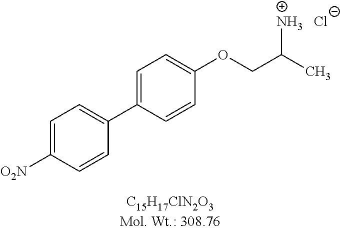 [SMILES: CC([NH3+])COc1ccc(-c2ccc([N+](=O)[O-])cc2)cc1 ; [Cl-]] | JME-209-2 |
| 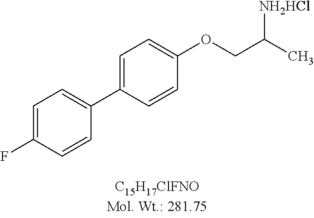 | JME-209-3 |
| 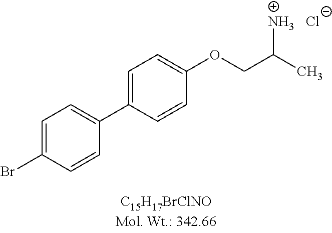 [SMILES: CC([NH3+])COc1ccc(-c2ccc(Br)cc2)cc1 ; [Cl-]] | JME-209-4 |
| 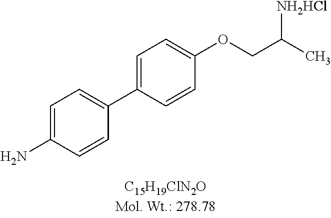 | JME-209-5 |
| 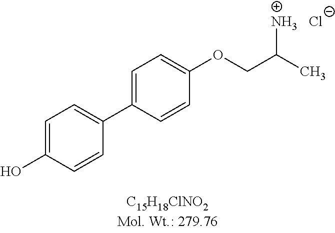 [SMILES: CC([NH3+])COc1ccc(-c2ccc(O)cc2)cc1 ; [Cl-]] | JME-209-6 |
| 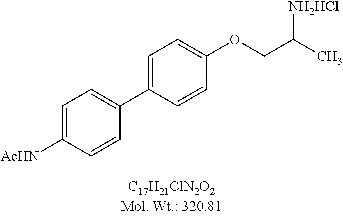 | JME-209-7 |
| 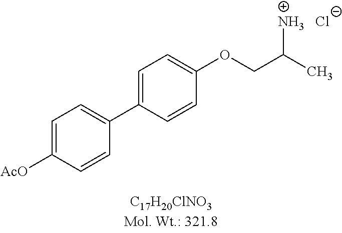 [SMILES: CC(=O)Oc1ccc(-c2ccc(OCC(C)[NH3+])cc2)cc1 ; [Cl-]] | JME-209-8 |

3. A pharmaceutical composition comprising at least one compound as defined in claim 1, and a pharmaceutically acceptable carrier.

4. The pharmaceutical composition according to claim 3, in a form of a tablet, a capsule, a powder for reconstitution, an oral solution, an oral suspension or an inhalation solution.

5. The pharmaceutical composition according to claim 4 in the form of the tablet, the capsule or the powder for reconstitution, which further comprises at least one agent selected from the group consisting of a disintegrant, a glidant, a binder, a diluent, a flavoring, a colorant, a sweetener and a lubricant.

6. The pharmaceutical composition according to claim 4 in the form of the oral solution, which further comprises at least one agent selected from the group consisting of an antioxidant, a preservative, a pH corrector, a flavoring, a colorant, a sweetener, a solubilizer and a solvent.

7. The pharmaceutical composition according to claim 4 in the form of the oral suspension, which further comprises at least one agent selected from the group consisting of a suspender, an antioxidant, a preservative, a pH corrector, a sweetener, a flavoring, a colorant and a solvent.

8. The pharmaceutical composition according to claim 4 in the form of the inhalation solution, which further comprises at least one agent selected from the group consisting of an isotonizing agent, a surfactant, a pH corrector and a solvent.

9. The pharmaceutical composition according to claim 3, wherein the at least one compound is represented by at least one of the structures below:

| Structure | Code |
|---|---|
| 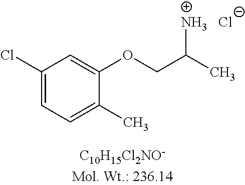 | JME-170 |
| 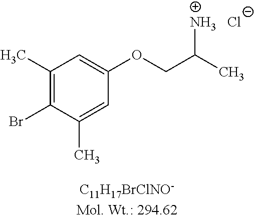 | JME-173 |
| 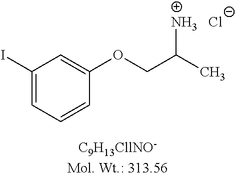 | JME-141 |
| 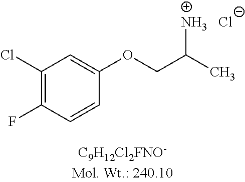 | JME-188 |
| 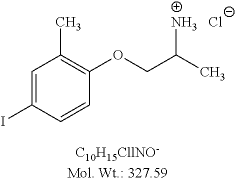 | JME-207 |
| 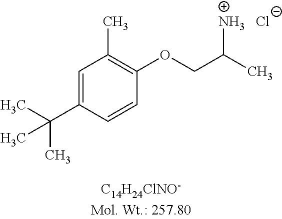 [SMILES: Cc1cc(C(C)(C)C)ccc1OCC(C)[NH3+] ; [Cl-]] | JME-208 |
| 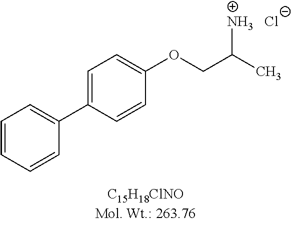 [SMILES: CC([NH3+])COc1ccc(-c2ccccc2)cc1 ; [Cl-]] | JME-209 |
| 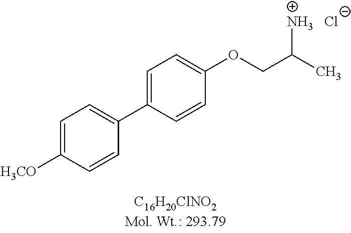 [SMILES: COc1ccc(-c2ccc(OCC(C)[NH3+])cc2)cc1 ; [Cl-]] | JME-209-1 |
| 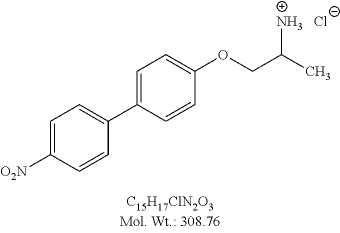 [SMILES: CC([NH3+])COc1ccc(-c2ccc([N+](=O)[O-])cc2)cc1 ; [Cl-]] | JME-209-2 |
| 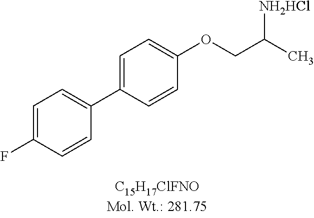 | JME-209-3 |
| 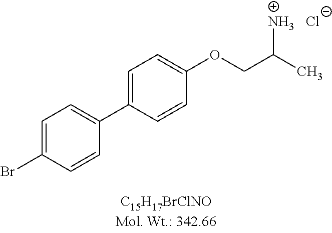 [SMILES: CC([NH3+])COc1ccc(-c2ccc(Br)cc2)cc1 ; [Cl-]] | JME-209-4 |
| 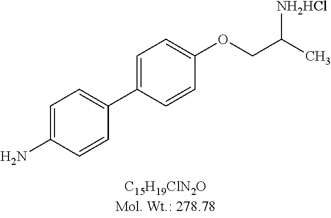 | JME-209-5 |
| 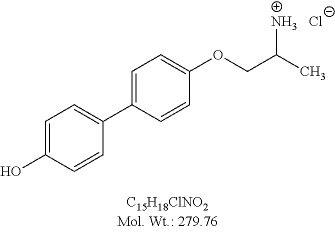 [SMILES: CC([NH3+])COc1ccc(-c2ccc(O)cc2)cc1 ; [Cl-]] | JME-209-6 |
| 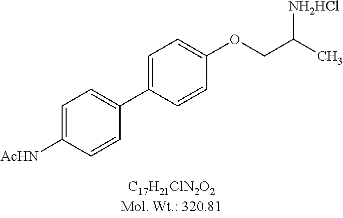 | JME-209-7 |
| 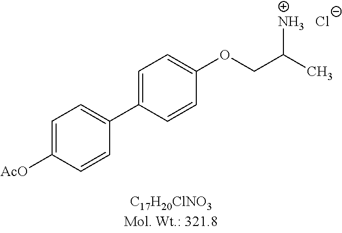 [SMILES: CC(=O)Oc1ccc(-c2ccc(OCC(C)[NH3+])cc2)cc1 ; [Cl-]] | JME-209-8 |

10. The pharmaceutical composition according to claim 3, which is effective for the treatment, prevention or inhibition of pulmonary inflammatory diseases.

11. A method of treatment, prevention or inhibition of pulmonary inflammatory disease wherein said method comprises administering a pharmacologically effective amount of the composition as defined in claim 3 through any route of administration.
